(19) United States
(12) Patent Application Publication
(10) Pub. No.: US 2025/0195604 A1
(43) Pub. Date: Jun. 19, 2025

(54) Compositions and Methods of Treatment of Certain Vasculitis Syndromes

(71) Applicant: Soligenix, Inc., Princeton, NJ (US)

(72) Inventors: Oreola Donini, Coquitlam (CA); Christopher Schaber, Princeton, NJ (US); Adam Rumage, Princeton, NJ (US); Richard Straube, Princeton, NJ (US)

(21) Appl. No.: 18/963,115

(22) Filed: Nov. 27, 2024

Related U.S. Application Data
(60) Provisional application No. 63/603,027, filed on Nov. 27, 2023.

(51) Int. Cl. A61K 38/08; A61P 1/04

(52) U.S. Cl. CPC A61K 38/08; A61P 1/04

(57) ABSTRACT

A method of reducing the incidence of mucocutaneous ulcers or vasculitis in an individual comprising administering an effective amount of dusquetide in a formulation, wherein the formulation comprises no excipients. More specifically, a method of treating oral, genital or cutaneous aphthous lesions in an individual by administering an effective amount of excipient-free dusquetide, wherein the individual has been diagnosed with Behçet's disease.

### RELATED APPLICATIONS

This application claims the benefit of priority from U.S. Provisional Application No. 63/603,027 filed on Nov. 27, 2023, the contents of which are hereby incorporated by reference herein.

### FIELD OF THE INVENTION

The invention relates to the field of peptide formulations for the treatment of ulcers and vasculitis related conditions.

### BACKGROUND OF THE INVENTION

Behçet's disease (BD) or Behçet's syndrome is a rarely occurring vasculitis, potentially involving both arteries and veins of all sizes. Its etiology is incompletely understood but it has significant prevalence along the “Silk Road,” particularly Turkey (80-370 cases per 100,000), Iran and Japan. Incidence in Europe and North America is significantly lower.

BD most commonly manifests with mucocutaneous disease including orogenital ulcers (˜97% of patients experience oral lesions and 60-90% of patients experience genital lesions). The oral lesions are similar to benign recurrent aphthous stomatitis and can vary in size and may scar. The oral ulcers may affect eating and swallowing. Genital ulcers may affect any part of the genitourinary tract and may take 1-2 weeks to heal and more frequently result in scarring (occurring in approximately ⅔ of patients). They are also prone to recurrence and becoming infected (Hatemi 2023). Skin lesions also occur, including pathergic reactions in which minor trauma can lead to poorly healing skin ulcers or lesions. Ocular involvement occurs in 50-70% of patients, particularly in the form of uveitis. Vascular involvement occurs in 15 to 40% of patients, with a high frequency of recurrence even in the presence of immunosuppressive therapy. Involvement of the nervous system occurs more rarely and when directly affected can be classified as either parenchymal or cerebral venous sinus thrombosis (Hatemi 2023). BD can result in significant morbidity and mortality (Nair 2017, Hatemi 2023).

The symptoms of BD can be variable and intermittent, resulting in delays in definitive diagnosis. Diagnosis is driven by a constellation of symptoms based on guidelines including the International Criteria for BD (ICBD 2006), the Internal Team for the revision of the ICBD (ITR-ICBD 2014) and the International Study Group Criteria (see table in Attachment 8.1), as there is no specific blood test or marker for the disease. BD is at least partially an inflammatory condition, and is usually most severe in young to middle aged adults (20-40 years), with symptom severity subsiding as the patient ages (Nair 2017).

The etiology of BD is believed to be a combination of genetic markers (eg, HLA-B51 is found in approximately 60% of BD patients, HLA ERAPI has been identified in genome wide association studies). Environmental factors, including triggers for flare ups, may include microbial exposure and cellular and humoral immunity, resulting in a pro-inflammatory cytokine cascade and increased levels of inflammatory markers. Both the relapsing/remitting course of the disease and the treatment response to immunosuppressive medications indicates that BD is likely an autoimmune/autoinflammatory disease.

The prevalence of BD in the United States is primarily estimated on the basis of a 2009 retrospective evaluation of the incidence and prevalence of BD in Olmsted County, Minnesota over 45 years (Calamia 2009). A total of 13 patients were identified, yielding a point prevalence of 5.2 per 100,000 in 2000 and an annual age and sex-adjusted incidence of 0.38 per 100,000 population (95% confidence interval 0.17-0.59). Using the upper confidence interval of the age and sex-adjusted incidence (0.59 per 100,000) would yield 2000 patients across the US in 2021 (population assumption: 332 million).

A more recent evaluation (Hammam 2022) collected information on all confirmed BD cases in patients of at least 18 years in the Rheumatology Informatics System for Effectiveness (RISE) Registry between 2014 and 2018. The RISE Registry captures all patients seen by participating US rheumatologists. In 2018, the RISE Registry had validated data from 1113 providers in 226 practices. This represented >30% of the US clinical rheumatology workforce. There were 1323 patients identified in the RISE database. Assuming an equivalent prevalence in the non-indexed providers, would yield a total patient population prevalence of 1323/0.3=4410 patients.

A meta-analysis of prevalence of BD on the basis of geographic area and study type (census vs. sample surveys) suggested a prevalence and 95% confidence interval for North America/Caribbean Islands of 3.8 (2.2, 6.8) (Maldini 2018). Utilizing the higher 95% confidence interval would suggest a patient population of at most 22,576 patients based on the 2021 US population of 332 million.

Although sample size estimates vary, even the most generous assumptions yield patient populations well below 200,000 patients and well within the definition of an orphan indication. There is no cure for BD and management of the disease is a life-long process responsive to the manifestations of symptoms over time. Organ involvement can develop over time after an initial presentation of mucocutaneous disease. Presently, there are few treatment options for patients.

Dusquetide is a 5-amino acid peptide which modulates the response of the innate immune system (Yu 2009, North 2016). It has been shown to suppress the formulation of oral ulcers while also decreasing the rate of infection (Kudrimoti 2016, Kudrimoti 2017). Dusquetide is administered as a 4-minute IV infusion twice weekly. Given the severity, psychological impact, and potential morbidity associated with genital ulcers, dusquetide administered twice weekly after a flare of genital lesions is thought to enhance the rate of resolution and to potentially decrease the rate of infection and scarring of genital lesions in BD.

There remains a need in the art to have a peptide formulation of therapeutic value for the treatment of certain conditions relating to ulcers or vasculitis, such as BD, which can be administered to the patient in a safe and effective manner.

### SUMMARY OF THE INVENTION

The present invention provides for a method of reducing the incidence of mucocutaneous ulcers or vasculitis in an individual comprising administering an effective amount of dusquetide in a formulation, wherein the formulation comprises no excipients.

In another embodiment, the present invention provides for a method of treating oral, genital or cutaneous aphthous lesions in an individual by administering an effective amount of excipient-free dusquetide, wherein the individual has been diagnosed with BD.

### BRIEF DESCRIPTION OF THE DRAWINGS

The following figures provide illustrative examples of the present invention and are incorporated by reference within this disclosure.

FIG. 1 shows in vitro LPS-stimulated TNF RII/TNFα and IL-1ra/IL-1b ratios of blood from SAD and MAD volunteers receiving low doses of dusquetide (0.15 to 2.0 mg/kg) or placebo. Solid lines indicate the mean response, dashed lines the respective 95% CI for the population. Boxes indicate non- or minimally-overlapping 95% CI between the 2 populations.

FIG. 2 shows median duration of severe oral mucositis (SOM; a form of aphthous ulcer) by treatment group in patients receiving 1.5 mg/kg of dusquetide compared to the placebo group.

FIG. 3 shows median duration of SOM by treatment group in patients receiving chemotherapy every third week.

FIG. 4 shows median duration of SOM by treatment group in the intent to treat (ITT) and per protocol (PP) populations of Study IDR-OM-02.

FIG. 5 indicates consistency between median duration (Days) of SOM in IDR-OM-01 (Phase 2) and IDR-OM-02 (Phase 3) clinical studies.

FIG. 6 shows dusquetide reduction in the severity of dextran sulphate sodium (DSS)-induced colitis as measured by endoscopy on Days 7, 14 and 21.

### DETAILED DESCRIPTION OF THE INVENTION

Dusquetide is an innate defense regulator (IDR), a class of short, synthetic peptides with a novel mechanism of action. Dusquetide is a 5 amino acid peptide with the following sequence: L-arginyl-L-isoleucyl-L-valyl-L-prolyl-L-alanine-amide and having the following chemical structure:

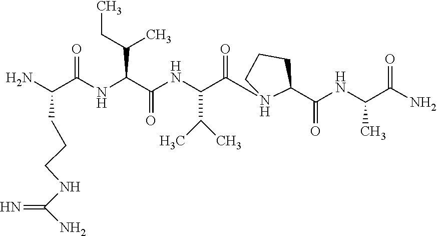 [SMILES: CCC(C)[C@H](NC(=O)[C@@H](N)CCCNC(=N)N)C(=O)N[C@H](C(=O)N1CCC[C@H]1C(=O)N[C@@H](C)C(N)=O)C(C)C]

The dosage form of dusquetide is an aqueous, aseptically processed, sterile solution for injection. Each vial contains 5 mL of a 60 mg/mL solution (300 mg of dusquetide). Dusquetide is formulated in water for injection and pH adjusted to a target value of 6.0. The preferred formulation of the present invention contains no excipients and has an osmolality of ˜300 mOsm/kg. Dusquetide drug product is diluted in sterile saline to the appropriate concentration for injection, determined on a mg/kg basis by the recipient's weight at a dose level of 1.5 mg/kg. Diluted dusquetide is administered as an intravenous (IV) infusion in 25 mL over 4 minutes, once every third day. In Phase 1 studies dusquetide has been safely administered at dose levels of 0.15 to 8.0 mg/kg.

BD has been increasingly associated with genetic abnormalities causing or concurrent with innate immune amplification (Shimizo 2023). Skin histopathology indicates neutrophilic dermatitis or panniculitis while pathergy tests in BD patients result in persistent lymphocyte and monocyte infiltrations compared to healthy individuals (Shimizu 2023). In both oral and genital lesions, lymphocyte infiltration is frequently observed in the lamina propria. In addition to neutrophil infiltration, macrophages have also been shown to have altered response profiles in response to BD sera, suggesting a prevalence of M1 type macrophages in BD patients (Shimizu 2023).

Similarly, impairment of M2 mediated anti-inflammatory function has been proposed as an underlying etiology of BD (Hirahara 2022). IL-1B, IL-6 and TNFα are all known to be elevated in serum from patients with BD (Li 2023, Shimuzu 2023). Dusquetide has been shown to alter the inflammatory response profile in response to lipopolysaccharide and other inflammatory stimulation, resulting in an increased anti-inflammatory response-specifically increasing ILIra relative to IL-1B and TNFRII relative to TNFα (North 2016, Yu 2009). Biological responses in oral aphthous ulcers formed in the context of chemotherapy and radiation treatment for cancer (oral mucositis) have been observed associated with clinical benefit after systemic administration of dusquetide (Kudrimoti 2016). Dusquetide is known to bind (but not inhibit) sequestome-1 (Yu 2009, Zhang 2022) which has been implicated in a number of innate immune pathways and macrophage M2 function. Based on these findings, dusquetide has the potential to be beneficial as a systemic treatment for the mitigation and enhanced resolution of genital ulcers in BD.

### EXAMPLES

Reduction in innate immune stimulation inflammation has been demonstrated in a Phase 1 study with ex vivo stimulation of blood samples with lipopolysaccharide (LPS). In a Phase 1 study, patients received single ascending doses (0.15 to 8.0 mg/kg) and multiple ascending doses (daily 7× 0.5 to 6.5 mg/kg) of dusquetide as 4-minutes IV infusions. To determine the pharmacodynamic profile of dusquetide, cytokine and chemokine analytes were quantified after 4 h of in vitro stimulation of whole blood with LPS (E. coli) to determine dusquetide effects on dose, gender and any extended impact on PD.

The following panel of cytokine and chemokine analytes were quantified: IL-la; IL-1B; IL-6; IL-1ra; TNFα; Tumor Necrosis Factor-Receptor II (TNF RII); MCP-1; Macrophage Inflammatory Protein-1alpha (MIP-la; also known as CCL3); MIP-1B (also known as CCL4) and RANTES (also known as CCL5). The dusquetide-treated population (N=56) in the combined SAD and MAD data set was subdivided into “low dose” (N=26, 0.15-2.0 mg/kg) and “high dose” (N=30; 3.0-8.0 mg/kg) groups and these were compared to the combined placebo group (N=22).

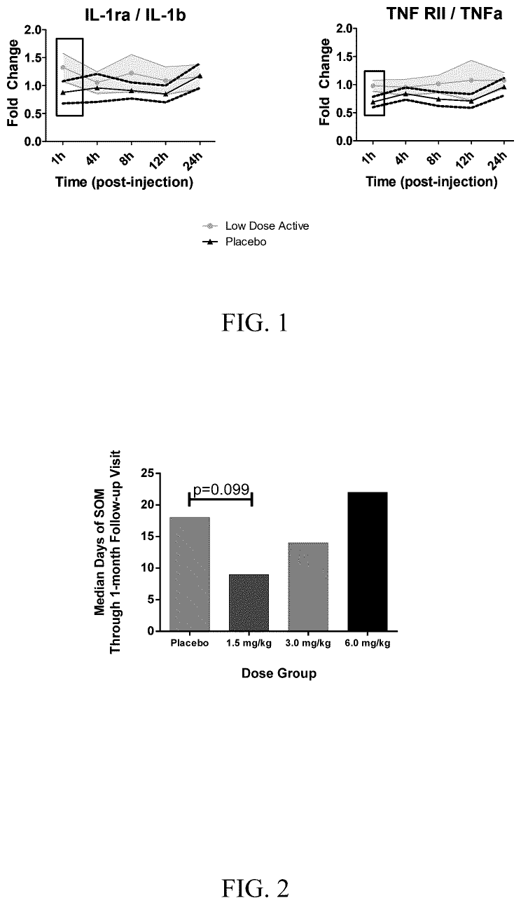

Evaluations of the combined SAD and MAD data sets showed that the analytes IL-1x, IL-1B and TNFα appeared to be slightly decreased while IL-1ra was slightly increased at the 1 h timepoint in the low dose group as compared to either the placebo or high dose groups. The ratios IL-1ra/IL-1B and TNF RII/TNFα in each subject showed a more distinct increased responses in the low dose group than the changes in either of the contributing analytes alone, suggesting that an increase in the “anti-inflammatory status” is observed in the low dose group (i.e., higher anti-inflammatory TNF RII and IL-1ra levels coupled with lower TNFα and IL-1β levels in each individual) (see FIG. 1). In general, the high dose group appeared to be very similar to the placebo group with respect to the measured analytes. Because dusquetide does not directly impact cytokine levels, but modulates them via other pathways, it is not unexpected that the response is not linearly dose responsive. The lack of response at higher dose levels of dusquetide likely reflects the induction of multiple control pathways, which in aggregate result in a lack of effect on cytokine/chemokine levels.

Reduction in the occurrence of oral aphthous ulcers has been observed in Phase 2 and Phase 3 studies of oral mucositis in head and neck cancer patients. The Phase 2 study was designed to examine the effects of dusquetide on ameliorating OM in patients with oral or oropharyngeal squamous cell carcinoma who were undergoing therapy with fractionated radiation (2.0-2.2 Gy per day for a total planned cumulative dose of 55-75 Gy) and chemotherapy with cisplatin given either as “low dose” (30-40 mg/m2 every week) or as “high dose” or “aggressive” cisplatin (80-100 mg/m2 given every 3 weeks). Dusquetide was evaluated in this dose ranging study at doses of 1.5, 3.0 (3 patients only) and 6.0 mg/kg.

Based on the Phase 1 data, a dose of 1.5 mg/kg was expected and confirmed to be the most efficacious. Patients receiving 1.5 mg/kg of dusquetide had a 50% reduction in their median duration of severe oral mucositis (SOM) compared to the placebo group (9 days versus 18 days for the 1.5 mg/kg and placebo groups, respectively), as shown at FIG. 2. This finding was supported by a 39% reduction in the median AUC for the WHO Grade-time calculation, and a 7% reduction in the incidence of SOM when compared to placebo. Dusquetide appeared to be most effective in subpopulations with the longest potential duration of SOM.

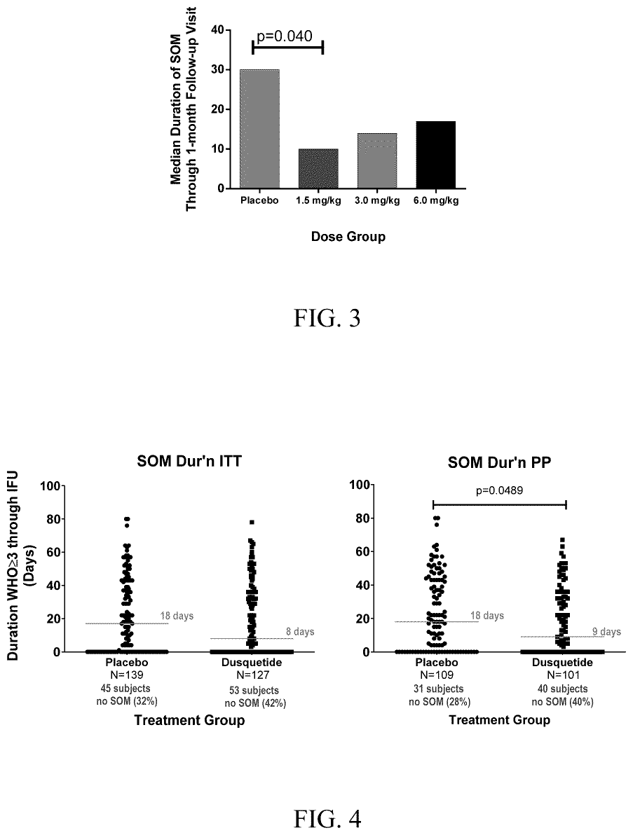

In the subpopulation of patients receiving every 3rd week cisplatin (i.e., aggressive cisplatin therapy), as indicated in FIG. 3, there was a 67% decrease in the median duration of SOM (from 30 to 10 days in the placebo and dusquetide 1.5 mg/kg dose groups, respectively), a 51% reduction in the median AUC WHO Grade-time calculation, and an 18% relative reduction in incidence of SOM when compared to the placebo, but no impact on the onset of disease.

In the complementary group of patients receiving weekly cisplatin and 1.5 mg/kg of dusquetide, the median duration of SOM in patients was decreased by 50% from 10 days in the placebo group to 5 days in the dusquetide group. This was associated with a 55% reduction in the AUC from 23.3 to 10.5 in the placebo and dusquetide 1.5 mg/kg dose groups, respectively. The reductions in oral aphthous lesions over time was also associated with a reduction in overall infection rate (Kudrimoti 2016), and although this reduction may have been due to the reduced lesion occurrence over time, it was also consistent with the observed anti-infective action of dusquetide (North 2016).

Subsequently a Phase 3 international study (Study IDR-OM-02) evaluated dusquetide in the treatment of oral aphthous lesions in head and neck cancer patients receiving concomitant chemoradiation therapy (at least 55 Gy radiation therapy and 80 to 100 mg/m2 cisplatin received every third week). 266 patients were enrolled in the randomized, blinded and placebo-controlled study assessing treatment with 1.5 mg/kg dusquetide administered twice per week as a 4-minute infusion. A statistically significant response was observed in the per protocol (PP) population, although not in the intent to treat (ITT) population (see FIG. 4).

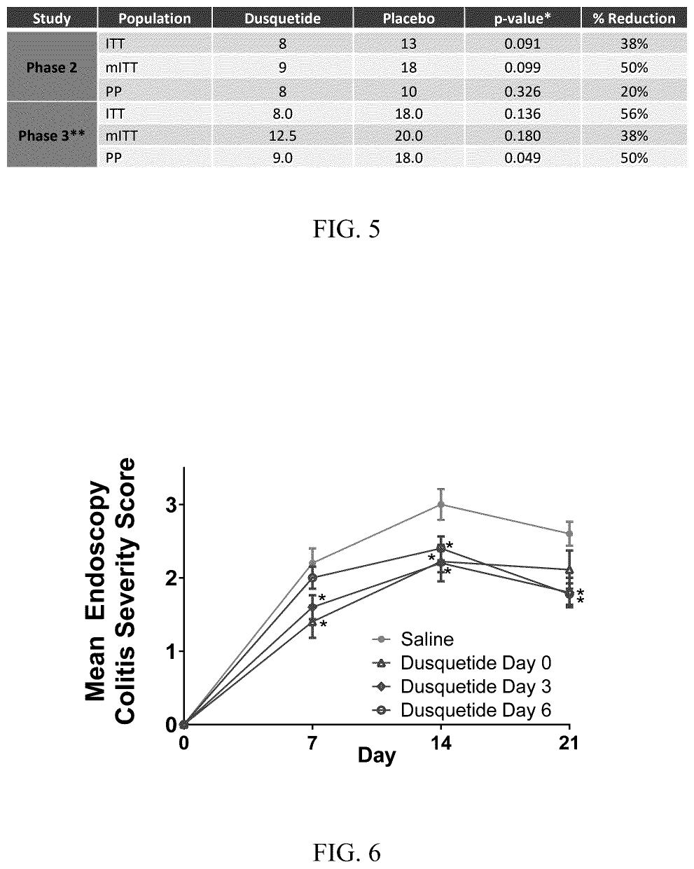

Results in the Phase 2 and Phase 3 oral mucositis studies were very consistent, as further detailed in FIG. 5.

Dusquetide binds to an intracellular adaptor protein, sequestosome-1, also known as p62, that is highly conserved between species and is involved in the efficient transmission of information during intracellular signal transduction, receptor trafficking and protein turnover (Yu 2009, Zhang 2022). p62 is known to function at a key nodal position in this signaling network, interacting with proteins downstream of Toll-like Receptors (TLRs) and Tumor Necrosis Factor Receptors (TNFRs). These receptors and the associated signaling network, including p62, are expressed in a wide variety of circulating, vascular wall and tissue-resident cell types.

Dusquetide binding to p62 selectively alters its interactions with other proteins such as RIP1 in these critical signaling cascades, while having no effect on p62 interactions with, e.g., PKCS and TRAF6 (Yu 2009). Studies in mouse macrophage-like and human epithelial cell lines have shown that, following exposure to dusquetide, transient activation occurs of mitogen activated protein kinase (p38 MAP kinase) and a transcription factor, CCAAT-enhancer binding protein (C/EBPB). Unlike the effect of drugs targeting the TLRs themselves, the binding of dusquetide to p62 does not cause persistent activation of NFKB, a transcription factor associated with potentially harmful inflammatory responses. Downstream of C/EBPβ, studies in mouse, nonhuman primate (NHP), and human primary cell cultures have shown that the expression patterns of chemokines such as monocyte chemotactic protein (MCP-1) are modulated by dusquetide exposure (North 2016).

The innate immune system involves numerous cell types distributed throughout the body in both circulating and tissue-resident compartments. The interaction of all the cells defines the aggregate response. Hence, the pharmacodynamic (PD) response to dusquetide is most appropriately characterized at a whole-body level. Nevertheless, cellular and molecular studies with individual cell types or sub-groups of cell types are informative but must be interpreted cautiously when used to predict the behavior of the system in vivo. For example, studies in mouse primary cell cultures have revealed that, when exposure to dusquetide occurs in vivo prior to culture of blood cells, the drug treatment causes an increase in the ex vivo MCP-1 response and a decrease in the ex vivo TNFα response to lipopolysaccharide (LPS; Gram-negative endotoxin) challenge. By contrast, when dusquetide exposure of either mouse or human blood occurs in vitro, although the TNFα response to LPS challenge is unaffected, the MCP-1 response is reduced. The responses of mouse and human primary blood cells to dusquetide in this in vitro setting are quantitatively and qualitatively similar.

The requirement for intercellular communication in the dusquetide mode of action has also been demonstrated by transfer of cell culture medium from primary mouse splenocytes to a mouse macrophage-like cell line. Incubation of dusquetide with the splenocytes, followed by transfer of the splenocyte culture medium (which no longer contains dusquetide) to a macrophage cell line culture, modulates the response of the macrophage cell line suppressing IL-10, IL-6 and TNFα (North 2016). This study demonstrates that the synchronized modulation of specific cytokine and chemokine responses observed with dusquetide in various contexts are secondary events after dusquetide treatment, and not the primary mediators of dusquetide action.

Dusquetide has been extensively studied in animal models of both mucosal damage (where damage associated molecular patterns (DAMPS)) trigger the innate defense system) and bacterial infection (where pathogen associated molecular patterns (PAMPS)) trigger the innate defense system). Dusquetide modulates the innate defense response to tissue injury, reducing the severity of damage caused by the inflammatory cascade and enhancing resolution of disease. Mucosal tissue was protected following chemotherapy in mice, as evidenced by the decreased intensity and duration of mucositis (both orally and in the lower gastrointestinal (GI) tract) (Kudrimoti 2016). Similarly, in a hamster model of fractionated radiation-induced mucositis, a mimic of the fractionated radiation therapy used in the treatment of HNC patients, dusquetide was found to decrease both intensity and duration of SOM and to be effective when administered every 2nd or 3rd day at 25 mg/kg.

In a mouse model of dextran sulphate sodium (DSS)-induced colitis, further protection of the lower GI was demonstrated (Kudrimoti 2016) at levels comparative to those reported to maximal steroid use. Studies in mouse in vivo models of bacterial infections have evaluated dusquetide administration at various dose levels and various times in relation to the time of bacterial challenge (PAMP triggering of the innate defense response) (North 2016). Such investigations demonstrate that IV or intraperitoneal (IP) administration of dusquetide provides increased survival and reduced bacterial growth in a number of Gram-positive and Gram-negative lethal infections in various tissues. These include methicillin resistant S. aureus (MRSA) bacteremia, intramuscular S. aureus, intramuscular MRSA, K. pneumoniae peritonitis, and B. pseudomallei sepsis. Dusquetide treatment also reduced the ulceration and swelling of abraded skin of mice exposed to MRSA.

Investigations of dosing requirements in an IV MRSA challenge model (bacteremia) in mice indicate that IV dusquetide can be administered either therapeutically or prophylactically and is complementary to antibiotic administration. Although the drug has a short circulating half-life, the PD effects of a single dose last up to 5 days in mice depending on the dose level administered. Daily dosing is therefore not required and dosing every third day is sufficient. Efficacy is consistently seen at IV doses greater than or equal to 5 mg/kg in mice; doses of 25 mg/kg are used routinely.

MRSA infection experiments in athymic nude mice show that T lymphocytes are not required for the survival benefit of dusquetide treatment. Consistent with these findings, investigations in mice rendered severely leukopenic by cyclophosphamide treatment show that dusquetide provides survival benefit following bacterial challenge. These observations suggest that dusquetide treatment may be effective in individuals who have high susceptibility to infection or innate immune dysregulation due to poorly functioning immune systems.

The proposed pharmacological mechanism of dusquetide is a selective modulation of the innate defense response to challenge by pathogen or damage-associated molecules, as distinct from direct activation of elements of the innate defense system independent of other stimuli. Consistent with this, very few changes have been observed in circulating cytokine and chemokine levels or in blood cell phenotypes following dusquetide administration in healthy animals or humans whose innate defense system has not been activated. In normal mice, no changes in cytokine or chemokine levels were detected in response to dusquetide. In NHPs, there were no substantial changes in the levels of most cytokines and chemokines, with the exception of Interleukin-1 receptor antagonist (IL-1ra) and MCP-1. IL-1ra, an anti-inflammatory cytokine, showed a marked but variable increase in response to dusquetide treatment. These observations are in keeping with the general anti-inflammatory biological effects observed with dusquetide. In addition, modest changes in the levels of MCP-1 were observed in NHP, with remarkable consistency between animals in timing of these transient effects. Following triggering of the innate defense system, the transcription factor C/EBPB is activated, selectively increasing expression of chemokines such as RANTES and IP-10 at the site of infection, while decreasing IL-6 and TNFα. At the whole animal level, efficacy is correlated with increases in macrophage numbers at disease sites.

Further studies have explored the ability of blood cells to respond to a pathogen (or pathogen-like) challenge in vitro after prior exposure to dusquetide in vivo—an “ex vivo” test system that provides information about the “responsiveness” of the innate defense system. The responsiveness of mouse and NHP innate defense systems have been characterized by measuring changes in cytokine and chemokine production following ex vivo challenge of blood cells with LPS. In general, no evidence of broad-spectrum activation was observed. After prior in vivo treatment with dusquetide, blood samples were cultured with LPS and evaluated for 58 (mouse) and 47 (primate) cytokines and chemokines; insignificant changes were found in the responses of most analytes. Small changes were observed in select cytokines including decreases in TNFα and increases in MCP-1 and MCP-3 in LPS-treated blood from mice treated with dusquetide compared to saline. In blood from dusquetide-treated NHP, a marked increase in IL-1ra response was observed, coupled with modest short term (1 to 4 h after dusquetide administration) decreases in IL-1β and MIP-la responses and longer term (12-24 h after dusquetide administration) mild elevations in these same cytokine responses.

The ability of dusquetide to modulate a pre-existing inflammatory cascade driven by tissue damage is illustrated by the DSS colitis model. In this model, DSS was administered as a 3% DSS solution in the drinking water of male C57BL/6 mice from Days 0 to 5 of the study. Colitis was monitored by video endoscopy on Days 7, 14 and 21. Dusquetide (25 mg/kg IV) was administered every third day from Days 0 to 18 (Q3d d0-18), from Days 3 to 18 (Q3d d3-18) or from Days 6 to 18 (Q3d d6-18). The results of the study are shown in FIG. 6. By Day 14, all 3 dusquetide treatment regimens demonstrated a statistically significant reduction in endoscopic colitis severity score. However, reduction in Day 7 scores was only observed in groups which had received at least 2 doses of dusquetide by that time (i.e., Q3d d0-18 and Q3d d3-18 but not Q3d d6-18). On Day 21, all 3 treatment groups appeared to be responding in a similar manner. Statistical analysis was undertaken using t-tests and an asterisk indicates statistically significant differences from control (p<. 05). The degree of response of this model was similar to that induced by 3 mg/kg prednisolone of 0.5 mg/kg budesonide, both administered to the mice at the upper limit of their tolerability.

As used in this specification and in the appended claims, the singular forms include the plural forms. For example, the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

We claim:

1. A method of treating or reducing the symptoms of a disease selected from the group consisting of Behçet's syndrome, ulcers, oral mucositis and thrombotic syndrome in a mammal by administering a formulation comprising an effective amount of a compound having the structure: 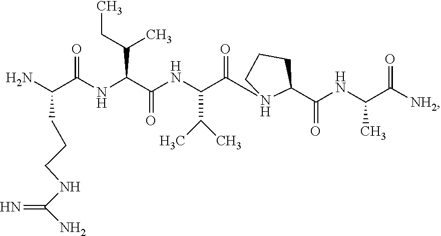 [SMILES: CCC(C)[C@H](NC(=O)[C@@H](N)CCCNC(=N)N)C(=O)N[C@H](C(=O)N1CCC[C@H]1C(=O)N[C@@H](C)C(N)=O)C(C)C]
wherein the compound administration also serves to increase the rate of ulcer healing in the mammal.

2. The method of claim 1, wherein the effective amount of the compound within the formulation is at least 1.5 mg/kg.

3. The method of claim 1, wherein the administration to the mammal is via a route selected from the group consisting of intramuscular, intraperitoneal, intravenous and subcutaneous.

4. The method of claim 1, wherein the formulation as administered contains no excipients.

5. The method of claim 2, wherein the formulation is administered twice per week as an infusion.

6. The method of claim 5, wherein the infusion is a 1 minute, 2 minute, 4 minute or up to 10 minutes in length.
